(19) United States
(12) Patent Application Publication
(10) Pub. No.: US 2011/0217201 A1
(43) Pub. Date: Sep. 8, 2011

(54) ABSORBENT PAD FOR TRASH RECEPTACLES

(76) Inventors: Ronald Jensen, Chicago, IL (US); Sayandro Versteylen, Ontario, CA (US)

(21) Appl. No.: 13/128,639

(22) PCT Filed: Nov. 11, 2009
(86) PCT No.: PCT/US2009/064073

§ 371 Date: May 10, 2011

Related U.S. Application Data
(60) Provisional application No. 61/113,506, filed on Nov. 11, 2008.

(51) Int. Cl. A61L 9/16; A61L 9/00

(52) U.S. Cl. 422/5; 422/120; 422/123

(57) ABSTRACT

The present disclosure relates to an absorbent pad with a layered architecture that is adapted for absorbing liquids in trash receptacles that masks or suppresses odors therein. The present disclosure also provides a method of using an absorbent pad to absorb trash liquids to reduce or mask odors, delay spoilage of food wastes, and repel rodents or other pests.

### BACKGROUND OF THE INVENTION

1. Field of the Invention

The present disclosure relates to an absorbent pad with a layered architecture that is adapted for absorbing liquids in trash receptacles that masks or suppresses odors therein. The present disclosure also provides a method of using an absorbent pad to absorb trash liquids to reduce or mask trash odors, delay spoilage of food wastes, and/or repel rodents and other pests.

2. Description of the Related Art

Trash receptacles, such as flexible trash bags, trash cans, and dumpsters, are commonly used to temporarily hold refuse in the home or at a place of business, until the refuse can be transported from the trash receptacle for permanent disposal at a landfill or by incineration.

Liquid wastes from beverages or edible liquids (such as oils and vinegar in a salad dressing) that are placed in the trash receptacle, as well as liquids generated by degradation of food wastes, can make trash odorous and unsanitary, accelerating spoilage of foods in the trash, and can cause a consumer to send the trash receptacle for permanent disposal long before the trash bag or trash can is completely full. Mixture of such liquids with other, non-edible liquids that may be placed in the same trash receptacle (such as those products or wastes that contain ammonia) may exacerbate the problem of odors in the trash. A flexible trash bag having a small rupture or leak that allows such accumulated trash liquids to escape will create a mess for a consumer.

In addition, trash liquids can support microbial growth and potential contamination of the trash receptacle, causing inconvenience and a potential safety hazard for a person who has to handle the trash. Trash liquids can degrade to produce chemicals associated with spoiled or rancid foods that can produce a disagreeable odor for a consumer at home, or for patrons of a restaurant or store when walking near a trash dumpster. Trash liquids and the odors they generate can also attract rodents, insects, and other pests to the trash receptacle and make handling of the trash unpleasant and unsanitary.

Because of these disadvantages, consumers often empty trash receptacles more often than would be necessary, but for the odor and spoilage of food wastes and liquids. For instance, consumers may dispose of a partially-filled trash bag because of odors or fear of attracting insects in the home, which is wasteful and inconvenient. Cumulatively, conventional trash management creates a large “carbon footprint” and creates additional environmental wastes.

### SUMMARY OF THE INVENTION

The present disclosure provides an absorbent pad having a layered architecture that is adapted for a trash receptacle for management of trash liquids by absorbing almost all liquids in the trash receptacle and reducing and/or masking trash odors, delaying spoilage of food wastes in the trash receptacle, and/or repelling rodents, insects, and other pests from the trash receptacle.

The present disclosure also provides an absorbent pad that is lightweight but can absorb a large amount of trash liquids, and is readily disposed with the trash. The absorbent pad can be made of a biodegradable material that will rapidly break down after the trash has been removed to a landfill for permanent disposal, reducing environmental waste and providing a smaller “carbon footprint.”

The present disclosure further provides that the absorbent pad of the present disclosure can be constructed to cover an interior surface of a trash receptacle. The absorbent pad can be secured to a trash receptacle, or simply placed inside a trash receptacle without being anchored in any manner.

The absorbent pad of the present disclosure can be packaged for use already inside of a trash receptacle, or can be packaged as an individual unit that is placed in a trash receptacle by a consumer at the time of use. The absorbent pad can be sized to fit in a flexible trash bag or trash can, or can be sized to cover the bottom of a dumpster.

The present disclosure also provides a method for reducing odors associated with trash liquids by placing an absorbent pad of the present disclosure in a trash receptacle. Another aspect of the present disclosure is a method for delaying food spoilage by placing an absorbent pad of the present disclosure in a trash receptacle. A further aspect of the present disclosure is a method of reducing environmental waste due to premature disposal of a flexible trash bag not filled to capacity, by using an absorbent pad of the present disclosure to absorb trash liquids therein.

### BRIEF DESCRIPTION OF THE DRAWINGS

FIG. 1 is an illustration of pad architecture of an exemplary embodiment of an absorbent pad of the present disclosure.

FIG. 2 is an illustration of pad architecture of another exemplary embodiment of an absorbent pad of the present disclosure.

FIG. 3 is an illustration of pad architecture of another exemplary embodiment of an absorbent pad of the present disclosure.

### DETAILED DESCRIPTION OF THE INVENTION

The present disclosure provides an absorbent pad having a layered architecture that is adapted for a trash receptacle for management of liquids in a trash receptacle (called “trash liquids” herein), and to reduce and/or mask trash odors, and/or delay spoilage of food wastes in a trash receptacle.

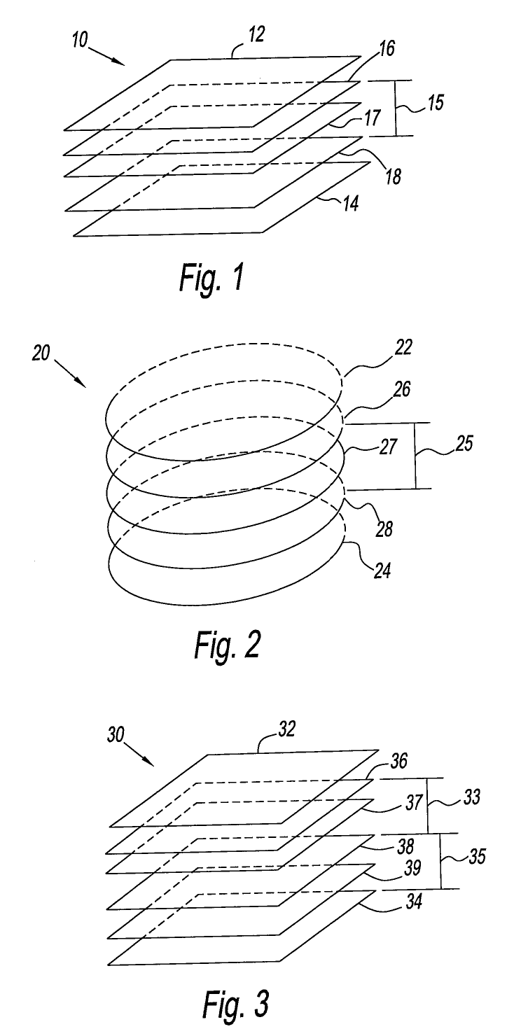

Referring now to the drawings, and, in particular, FIG. 1, there is provided an exemplary embodiment of an absorbent pad of the present disclosure, generally represented by reference number 10. Absorbent pad 10 has an architecture having a bottom layer 14, and two or more absorbent layers 16, 18. An absorbent layer 18 is positioned adjacent to bottom layer 14. Absorbent layers 16, 18 are made of one or more layers of an absorbent material. In this embodiment, absorbent layer 16 and absorbent layer 18 are an adjacent pair of absorbent layers that form pocket 15 therebetween. One or more agent 17 is positioned in pocket 15. The absorbent pad can further have a top layer 12. Top layer 12 and/or bottom layer 14 can be liquid permeable.

The absorbent material used in the two or more absorbent layers 16, 18 is able to absorb large quantities of trash liquids in a trash receptacle. The absorbent material can include, but is not limited to: cellulose, cellulose fiber, cellulose fluff, tissue, crepe tissue, paper, and any combinations thereof. If additional absorbency is required, a superabsorbent material, including, but not limited to, superabsorbent polymer (SAP) and compressed SAP composite material, can be incorporated in the pad architecture, including: between absorbent layers 16, 18; between top layer 12 and an adjacent absorbent layer; and/or between bottom layer 14 and an adjacent absorbent layer.

The absorbent material used for absorbent layers 16, 18 can vary as to: number of layers of absorbent material, type of absorbent material, treatment with surfactants, and/or overall thickness of the absorbent material, to selectively regulate the speed of liquid uptake and absorbency by absorbent layers 16, 18, as well as regulate the speed at which absorbed liquids will contact the one or more agent 17. For example, absorbent pad 10 can have absorbent layer 16 adjacent top layer 12 that is made of two layers of absorbent tissue, and absorbent layer 18 adjacent bottom layer 18 that is made of four layers of absorbent tissue, thereby providing rapid contact of liquids absorbed via top layer 12 and absorbent layer 16 with one or more agent 17, and slower, delayed contact of liquids absorbed via bottom layer 14 and absorbent layer 18 with one or more agent 17.

Top layer 12 can be made of a nonwoven material, or of a film made of polyethylene, polypropylene, and polyester, and/or any combinations thereof. The top layer can be treated or coated with a surfactant to enhance liquid permeability, and/or can be microperforated, to permit liquids to penetrate through the top layer to the absorbent layers.

Bottom layer 14 can be made of polyethylene, polypropylene, and polymer films, nonwoven, and any combinations thereof. The bottom layer can be liquid permeable or liquid impermeable. The bottom layer can be treated or coated with a surfactant to enhance liquid permeability, and/or can be microperforated to permit liquids to penetrate through the bottom layer to the absorbent layers.

Either top layer 12 and/or bottom layer 14 may be made of a polyethylene material that is rapidly degradable, and biodegradable, when exposed to heat, ultraviolet (UV) light, mechanical stress and/or moisture, to reduce the environmental impact of disposal of the absorbent pad.

Agent 17 can be one or more of the following: deodorizer, antimicrobial, animal or insect repellent, rodenticide, pesticide, bactericide, fungicide, virucide, disinfectant, sanitizer, sterilizer, mildewstat, and any combinations thereof.

In a first embodiment, agent 17 includes sodium bicarbonate (baking soda) as a deodorizer. Sodium bicarbonate reduces trash odors by chemically neutralizing acidic liquids present in trash liquids, such as vinegar, that can otherwise cause unpleasant odors and accelerate decomposition of food wastes in the trash. Sodium bicarbonate can also physically bind to certain odor-causing chemicals to reduce odor perception near the trash receptacle. Other embodiments employ an agent 17 that chemically neutralizes basic liquids present in trash liquids, such as ammonia, that can cause unpleasant odors near the trash receptacle and accelerate decomposition of food wastes. Still another embodiment has one or more agent 17 that chemically interacts with, and neutralizes, both acidic compounds and basic compounds present in trash liquids to reduce trash odor.

In another embodiment, agent 17 can be a masking agent that masks a trash odor. The masking agent can be a fragrance or other pleasant scent that masks unpleasant trash odors without necessarily chemically neutralizing or binding the compounds causing the trash odors. The masking agent can be a fragrance that includes, but is not limited to, one or more of the following: pine, peppermint, spearmint, vanilla, citrus (e.g., lemon, orange, or grapefruit), and/or florals (e.g., lavender or rose), and any combinations thereof.

Another embodiment has one or more agent 17 that is an antimicrobial agent, to inhibit the growth of microorganisms in trash liquids and/or on the surface of food wastes in the trash, thereby delaying spoilage of food wastes. Examples of the one or more antimicrobial agent include, but are not limited to: metal, metal compound, surface active agent, quaternary ammonium compound, organic acid, inorganic acid, salt, sulfite, polymer (including synthetic polymer and biopolymer), chitin, chitosan, nisin, enzyme, arginate, diacetate, antioxidant, or any combinations thereof. Other embodiments employ one or more agent 17 that is an oxygen scavenging enzyme, such as oxidase (including glucose oxidase and hexose oxidase), oxygenase, peroxidase, oxidoreductase, invertase, amylase, catalase, maltase, and any combinations thereof.

Another embodiment includes one or more agent 17 that is a rodenticide. In an exemplary embodiment, a rodenticide is positioned in pocket 15, so the rodenticide is not exposed unless the pocket is ripped open by a rodent biting into the pocket, killing the rodent after contact. In another embodiment, the one or more agent 17 is a rodent repellent that repels any rodent that is near or inside the trash receptacle. These embodiments reduce inconvenience and increase the safety of a person who cleans up the trash after its disruption by a rodent.

Another embodiment has one or more agent 17 that is an insecticide and/or an insect repellent, to repel or kill an insect, such as a fly, that is near (or in) the trash receptacle. Likewise, other embodiments have agents 17 that repel or kill other types of animal pests may be selected, depending on the needs at a particular location.

Agent 17 can be either bound, or unbound, to absorbent material in absorbent layers 16, 18 that form pocket 15. In another embodiment, agent 17 is incorporated into a laminate layer with an absorbent or superabsorbent material, which is incorporated into the pad architecture.

Agent 17 can be positioned in pocket 15 in its “active” form; that is, a form of agent 17 that is able to exert an intended effect (such as reducing or masking trash odors, or sanitizing trash liquids) without requiring further chemical or physical transformation by another component. In another embodiment, agent 17 can be present in absorbent pad 10 in an “inactive” form; i.e., a form that requires further chemical or physical transformation by interaction with another component to exert an intended effect.

FIG. 2 illustrates another exemplary embodiment of an absorbent pad of the present disclosure, generally represented by reference number 20, having a rounded shape for better fit in certain trash receptacles, such as a round trash can. Absorbent pad 20 has an architecture having a top layer 22 and a bottom layer 24 that are separated by two or more absorbent layers 26, 28. Pocket 25 is formed by an adjacent pair of absorbent layers 26, 28. One or more agent 27 is disposed in pocket 25. Absorbent layer 26 and absorbent layer 28 can each be made of one or more layers of an absorbent material. The rate of liquid uptake and absorbency by absorbent layers 26, 28 can be selectively regulated by the number of layers of absorbent material, type of absorbent material, treatment with surfactants, and/or overall thickness of the absorbent material used for each of absorbent layer 26 and absorbent layer 28.

FIG. 3 provides another exemplary embodiment of an absorbent pad of the present disclosure, generally represented by reference number 30. Absorbent pad 30 has an architecture having a top layer 32, a bottom layer 34, and absorbent layers 36, 38 positioned therebetween. Absorbent layer 36 and adjacent absorbent layer 38 form a first pocket 33. A first agent 37 can be positioned in first pocket 33. Bottom layer 34 and an adjacent absorbent layer 38 form a second pocket 35. A second agent 39 can be positioned in second pocket 35. Absorbent pad 30 has a first agent and a second agent that are physically separated from each other in the pad architecture. Other embodiments of absorbent pad 30 (not shown) can have more than two pockets in the pad architecture, such as three pockets, four pockets, or five pockets, in which additional agents can be separately positioned away from other agents in the pad architecture.

FIGS. 1 to 3 each illustrate the general ordering of layers in pad architectures, but do not represent the actual size, shape, or form of a particular layer. For example, FIG. 1 illustrates an embodiment having absorbent layers 16 and 18 as an “adjacent pair” of absorbent layers that form a pocket 15, in which an agent 17 is positioned; however, absorbent layers 16, 18 remain adjacent each other.

Another exemplary embodiment of an absorbent pad of the present disclosure has an absorbent pad with an architecture having a bottom layer, and two or more absorbent layers, with one absorbent layer positioned adjacent to the bottom layer. In this embodiment, an adjacent pair of the two or more absorbent layers forms a pocket. An agent is positioned in the pocket. This embodiment can further have a top layer that is positioned above the two or more absorbent layers. The absorbent pad absorbs almost all liquids in the trash receptacle, and mitigates trash odor and/or delays spoilage of food wastes in the trash receptacle.

Another exemplary embodiment of an absorbent pad of the present disclosure has an architecture having a top layer, and two or more absorbent layers, with one absorbent layer positioned adjacent to the top layer. In this embodiment, an adjacent pair of the two or more absorbent layers forms a pocket. An agent is positioned in the pocket. This embodiment can further have a bottom layer positioned below the two or more absorbent layers. The absorbent pad absorbs almost all liquids in the trash receptacle, and mitigates trash odor and/or delays spoilage of food wastes in the trash receptacle.

In each of the exemplary embodiments above, the top layer can be made of a nonwoven material, or of a film selected from the group consisting of polyethylene, polypropylene, and polyester, and any combinations thereof. The bottom layer can be selected from the following: polyethylene, polypropylene, polyester, nonwoven, and any combinations thereof. The top layer and/or the bottom layer can be treated or coated with a surfactant to enhance liquid permeability, and/or can be microperforated, to permit liquids to penetrate to the absorbent layers.

Scaling, or selecting, the proper amounts of an agent to employ in an absorbent pad of the present disclosure starts with the amount of absorbent material in the two or more absorbent layers, as well as the expected amounts of trash liquids to be absorbed (e.g., some pad architectures can be adapted for “heavy duty” absorption of trash liquids). In the present disclosure, the absorbent pad can have two or more absorbent layers that are “scaled” to the amount of an agent that is positioned in a pocket that is formed by an adjacent pair of the two or more absorbent layers. If an absorbent pad architecture uses too much absorbent material relative to the expected amounts of trash liquids, there may be insufficient liquid to dissolve or otherwise activate an agent; conversely, an absorbent pad that employs too little absorbent material for placement in a trash receptacle having a large quantity of trash liquids can “drown” or dilute the agent to impair its effectiveness. Scaling the amounts of the absorbent layers in relation to the amount of the agent(s) in an absorbent pad of the present disclosure has a beneficial effect on reducing trash odors and/or delaying spoilage of food wastes in trash receptacles.

A “pocket” (also called “island” interchangeably) means a space formed between two adjacent layers in the absorbent pad that can hold an agent in position in the pad architecture. A pocket can be formed between two adjacent absorbent layers, between a top layer and an adjacent absorbent layer, and/or between a bottom layer and an adjacent absorbent layer. A pocket can also protect an agent from being prematurely activated and used up by contact with ambient moisture in the air, as well as by premature contact with another agent also present in the absorbent pad.

The “architecture” of an absorbent pad of the present disclosure means the particular structure and order of absorbent material and agents positioned therein. The architecture of the absorbent pad affects performance when absorbing liquids in the trash receptacle; that is, the speed and amount of liquid uptake, and activity of agent(s) in the pad depends on their particular arrangement and positions in the absorbent pad, and not just on the size and quantity of absorbent material and agent(s) placed therein. The position of an active agent in relation to the absorbent tissue layers (and how near to the top or bottom layers of the absorbent pad) may affect how quickly or slowly the agent is activated by contact with liquids in the trash receptacle. Likewise, the particular arrangement of one group of absorbent layers in relation to another group of absorbent layers can affect the speed of liquid uptake by the absorbent pad. In this way, two absorbent pads that have the same number of absorbent layers and the same active agent, but having different pad architectures, may perform differently from each other when placed in a trash receptacle.

The absorbent pad of the present disclosure can also have a securing device (not shown) that secures the absorbent pad to a trash receptacle. The securing device can be disposed on an outer surface of an absorbent pad, on an interior surface of a trash receptacle, or a combination of these (e.g., having a hook-and-eye system such as VELCRO® with a first piece positioned on the absorbent pad, and the opposite piece positioned on the trash receptacle).

In an alternative embodiment, the absorbent pad of the present disclosure is simply placed inside a trash receptacle by the consumer, and is not secured in any way inside of a trash receptacle.

The securing device can be an adhesive material, including, but not limited to, two-sided tape, biodegradable adhesives, removable tacky adhesive, glue, epoxy, cements, or any combinations thereof. Alternatively, the securing device can be rails, hooks, or latches on an interior surface of a trash receptacle that secures an absorbent food pad thereto. The securing device may be a material that includes, but is not limited to, plastic, rubber, polymers, composites, or any solid material. The securing device can be an integral part of a trash receptacle, or, alternatively, can be permanently, or removably, attached to the trash receptacle by an adhesive material. In an exemplary embodiment, an absorbent pad of the present disclosure is secured by an adhesive material on an outer surface of the absorbent pad to an interior surface of a flexible trash bag. In a second embodiment, an absorbent pad of the present disclosure is secured to an interior surface of a trash can having rigid side walls and a bottom side. In another embodiment, the absorbent pad is secured by an adhesive material, such as two-sided tape, to an interior surface of a large trash receptacle, such as the bottom side of a large trash dumpster.

An absorbent pad of the present disclosure can be secured to, or positioned along, any interior surface of a trash receptacle, such as the bottom, side walls, and/or lid of a trash receptacle. The absorbent pad does not need to be precisely matched in shape or contour to an interior surface of the trash receptacle, as the addition of trash in the receptacle will press the pad outward to conform to the interior surfaces of the trash receptacle. The absorbent pad is pliable to conform more closely to the sides of the trash receptacle. An embodiment of the absorbent pad has a base panel that is connected to one or more side panels, where the side panels are connected to an edge of the base panel as by a hinge, to cover the bottom and side walls of a trash receptacle.

An absorbent pad of the present disclosure can be sized to fit various types of trash receptacles, ranging from small absorbent pads for flexible trash bags to very large absorbent pads adapted for placement in large trash dumpsters, such as are found outside of restaurants, fast food preparers, gas stations, and convenience stores. Because of exposure to sun, heat, and inclement weather, food wastes placed in dumpsters are particularly susceptible to decomposition that can create an unpleasant odor outside of the restaurant or business, where customers are walking. An absorbent pad sized for a dumpster can reduce the odors and delay spoilage of food wastes in the dumpster by absorbing almost all trash liquids in the absorbent material, and neutralizing or masking odors with agents such as sodium bicarbonate or a mint scent, and/or delaying spoilage with an antimicrobial agent. A rodenticide or rodent repellent can decrease disruption of the trash in the dumpster by rodents. The overall effects create a healthier, safer, and more pleasant environment near a trash dumpster.

As used herein, “dumpster” means a very large capacity trash receptacle (also called a garbage bin) having a bottom side, four side walls, and one or more lids connected by a hinge to cover the top. Dumpsters typically have a capacity in excess of 95 gallons, and are made of metal or heavy-duty plastic. A “trash can” or “trash container” means a trash receptacle having a bottom and sidewalls (and optionally a fitted lid) that is a size and weight that can be moved and emptied by one person. A trash can typically has a capacity from about 3 gallons to about 60 gallons, and can be made of plastic or metal. Trash cans can be any shape, and adapted for indoor or outdoor use. A “flexible trash bag” means a pliable plastic bag, often made of polyethylene, that is used to hold and transport trash for permanent disposal. Flexible trash bags typically have a capacity from 2 gallons to 60 gallons or more, with common consumer sizes of 13 gallons (kitchen) and 30-33 gallons (general purpose). A flexible trash bag may be used alone, or as a liner for a similarly-sized trash can, and, after filling, can be removed for transport for permanent disposal.

For the convenience of the consumer, an absorbent pad of the present disclosure can be packaged for use already placed inside of a trash receptacle, or can be packaged as an individual unit that is placed in a trash receptacle by a consumer at the time of use. The absorbent pad can be sized to fit in a flexible trash bag or trash can, or can be sized to cover the bottom of a dumpster. As an exemplary embodiment, an absorbent pad of the present disclosure is placed inside of a flexible trash bag (secured or unsecured to the flexible trash bag) before being packaged for sale to a consumer. Alternatively, the absorbent pad of the present disclosure can be packaged separately from the flexible trash bags, included as a separate item within the same package as the flexible trash bags, or attached as a value-added item to a package of flexible trash bags.

An absorbent pad of the present disclosure may be packaged individually, or in packages containing multiple absorbent pads. Absorbent pads that are part of multi-pad packages may be packaged in a continuous roll or layers, with a line of perforations or cut-lines for the consumer to easily separate an individual absorbent pad to place in a trash receptacle.

A method is provided for reducing or mitigating odors caused by a trash liquid in a trash receptacle using an absorbent pad of the present disclosure. The method includes placing the absorbent pad in a trash receptacle to absorb the trash liquid. The absorbent pad has an architecture comprising: a bottom layer; two or more absorbent layers with one absorbent layer positioned adjacent the bottom layer and with the two or more absorbent layers being of an absorbent material; an adjacent pair of said two or more absorbent layers forming a pocket; and an agent to chemically neutralize and/or mask odors. The agent is positioned in the pocket. The absorbent pad can also have a top layer positioned above the two or more absorbent layers. Either, or both, of the top layer and the bottom layer can be liquid permeable. The absorbent pad absorbs almost all liquids in the trash receptacle, and mitigates trash odor in the trash receptacle.

Another aspect of the present disclosure is a method for delaying spoilage of food wastes from a liquid in a trash receptacle including placing an absorbent pad of the present disclosure in a trash receptacle. The absorbent pad has an architecture comprising: a bottom layer; two or more absorbent layers with one absorbent layer positioned adjacent the bottom layer and with the two or more absorbent layers being of an absorbent material; an adjacent pair of the two or more absorbent layers forming a pocket; and an agent that inhibits microbial growth in the liquid. The agent is positioned in the pocket. The absorbent pad can further include a top layer positioned above the two or more absorbent layers. The top layer and/or the bottom layer can be liquid permeable. The absorbent pad absorbs almost all liquids in the trash receptacle, and reduces spoilage of food wastes in the trash receptacle.

A further aspect of the present disclosure is a method of reducing environmental waste due to premature disposal of a flexible trash bag not filled to capacity using an absorbent pad of the present disclosure to absorb trash liquids. The method includes placing an absorbent pad in a flexible trash bag to absorb trash liquids therein. The absorbent pad has an architecture comprising: a bottom layer; two or more absorbent layers with one absorbent layer positioned adjacent the bottom layer and with the two or more absorbent layers being of an absorbent material; an adjacent pair of the two or more absorbent layers forming a pocket; and an agent positioned in the pocket. The agent is selected from: deodorizer, antimicrobial, animal or insect repellent, rodenticide, pesticide, bactericide, fungicide, virucide, disinfectant, sanitizer, mildewstat, and any combinations thereof. The absorbent pad can further include a top layer. The absorbent pad can be made of rapidly-biodegradable materials. The absorbent pad mitigates trash odor and/or delays spoilage of food wastes by absorbing almost all of the trash liquids in the flexible trash bag, permitting greater filling of the flexible trash bag prior to permanent disposal.

An advantage of using an absorbent pad of the present disclosure is that the absorbent pad can absorb trash liquids, reduce and/or mask odors, and potentially delay spoilage of food wastes in the trash. This provides a healthier and more pleasant environment in the home and business. The absorbent pad also increases the likelihood that a consumer will fill have a longer time to fill an individual trash receptacle, reducing costs and environmental waste, and reducing the consumer's “carbon footprint” for trash disposal.

As used herein, the term “trash liquid” includes any liquid present in a trash receptacle. A trash liquid can be exudates from solid or semi-solid foods placed in a trash receptacle, including poultry, beef, pork, vegetables, fruits, ice cream, but also any water or other fluid generated by the breakdown and decomposition of food wastes in the trash. Other trash liquids can include edible liquid foods such as vegetable oil and vinegar, as well as potable liquids such as sodas and water, that are placed in the trash. Trash liquids can also include non-food liquids, such as cleaners, sprays, and animal or human wastes in a trash receptacle. Trash liquids can produce an odor of their own, and also can serve as a food source for microbes that cause more rapid decomposition of foods and an increase in odor-generating chemical compounds produced by the decomposition reactions.

The disclosure and embodiments described herein are exemplary, and should not be construed as implying any particular limitation on the present disclosure. It should be understood that various alternatives, combinations and modifications could be devised by those skilled in the art. The present disclosure is intended to embrace all such alternatives, modifications and variances that fall within the subject matter disclosed herein.

What is claimed is:

1. An absorbent pad for a trash receptacle to absorb trash liquids, mitigate trash odors and/or delay spoilage of food wastes in the trash receptacle, said absorbent pad having an architecture comprising:
a bottom layer;
two or more absorbent layers, with one absorbent layer positioned adjacent said bottom layer, said two or more absorbent layers being of an absorbent material;
an adjacent pair of said two or more absorbent layers forming a pocket;
an agent positioned in said pocket,
wherein said absorbent pad absorbs almost all liquids in the trash receptacle, and mitigates trash odor and/or delays spoilage of food wastes in the trash receptacle.

2. The absorbent pad according to claim 1, wherein said bottom layer is selected from the group consisting of: polyethylene, polypropylene, polyester, nonwoven, and any combinations thereof.

3. The absorbent pad according to claim 1, further comprising a top layer, wherein said top layer is selected from the group consisting of: nonwoven, polyethylene, polypropylene, polyester, and any combinations thereof; and wherein said top layer is treated, coated, and/or microperforated to enhance liquid permeability.

4. The absorbent pad according to claim 1, wherein said absorbent pad is rapidly biodegradable when placed in a landfill.

5. The absorbent pad according to claim 1, wherein said agent is selected from the group consisting of: deodorizer, antimicrobial, animal or insect repellent, rodenticide, pesticide, bactericide, fungicide, virucide, disinfectant, sanitizer, sterilizer, mildewstat, and any combinations thereof.

6. The absorbent pad according to claim 5, wherein said deodorizer chemically neutralizes an odor-producing chemical in trash liquids to reduce trash odor.

7. The absorbent pad according to claim 1, wherein said agent comprises a masking agent that masks a trash odor.

8. The absorbent pad according to claim 7, wherein said masking agent is a fragrance selected from the group consisting of: mint, pine, vanilla, citrus, floral, and any combinations thereof.

9. The absorbent pad according to claim 1, wherein said agent is an active agent that repels or kills a rodent near or in the trash receptacle.

10. The absorbent pad according to claim 9, wherein said agent is an active agent that repels or kills said rodent when said rodent bites into said pocket and contacts said active agent.

11. The absorbent pad according to claim 1, wherein said agent is an active agent that repels or kills an insect near or in the trash receptacle.

12. The absorbent pad according to claim 1, wherein said agent is unbound to said absorbent material.

13. The absorbent pad according to claim 1, wherein said agent is formed in a laminate layer with said absorbent material.

14. The absorbent pad according to claim 1, further comprising a securing device that secures said absorbent pad to the trash receptacle.

15. The absorbent pad according to claim 14, wherein a part of said securing device is disposed on an outer surface of said absorbent pad.

16. The absorbent pad according to claim 1, wherein the trash receptacle is selected from the group consisting of: flexible trash bag, trash can, and dumpster.

17. A method for mitigating odor caused by a trash liquid in a trash receptacle, comprising:
placing an absorbent pad in the trash receptacle to absorb the trash liquid, said absorbent pad having an architecture comprising:
a bottom layer;
two or more absorbent layers, with one absorbent layer positioned adjacent said bottom layer, said two or more absorbent layers being of an absorbent material;
an adjacent pair of said two or more absorbent layers forming a pocket; and
an agent to chemically neutralize and/or mask odors, wherein said agent is positioned in said pocket,
wherein said absorbent pad absorbs almost all liquids in the trash receptacle, and mitigates trash odor in the trash receptacle.

18. The method according to claim 18, wherein said absorbent pad further comprises a top layer positioned above said two or more absorbent layers.

19. A method for delaying spoilage of food wastes from a liquid in a trash receptacle, comprising:
placing an absorbent pad in the trash receptacle to absorb said liquid, said absorbent pad having an architecture comprising:
a bottom layer;
two or more absorbent layers, with one absorbent layer positioned adjacent said bottom layer, said two or more absorbent layers being of an absorbent material; and
an adjacent pair of said two or more absorbent layers forming a pocket; and
an agent that inhibits microbial growth in said liquid, wherein said agent is positioned in said pocket,
wherein said absorbent pad absorbs almost all liquids in the trash receptacle, and reduces spoilage of food wastes in the trash receptacle.

20. A method for reducing environmental waste due to premature permanent disposal of a flexible trash bag that is not filled to capacity due to trash odor or unsanitary appearance of said flexible trash bag, comprising:
placing an absorbent pad in said flexible trash bag to absorb trash liquids in said flexible trash bag, said absorbent pad having an architecture comprising:
a bottom layer;
two or more absorbent layers, with one absorbent layer positioned adjacent said bottom layer, said two or more absorbent layers being of an absorbent material; and
an adjacent pair of said two or more absorbent layers forming a pocket; and
an agent positioned in said pocket, wherein said agent is selected from the group consisting of: deodorizer, antimicrobial, animal or insect repellent, rodenticide, pesticide, bactericide, fungicide, virucide, disinfectant, sanitizer, sterilizer, mildewstat, and any combinations thereof;
wherein said absorbent pad is biodegradable, and
wherein said absorbent pad mitigates trash odor and/or delays spoilage of food wastes by absorbing almost all of said trash liquids in said flexible trash bag, permitting greater filling of said flexible trash bag prior to permanent disposal.
